(19) United States
(12) Patent Application Publication
(10) Pub. No.: US 2021/0330229 A1
(43) Pub. Date: Oct. 28, 2021

(54) METHOD AND DEVICE FOR ATTENTION TRAINING, AND COMPUTER READABLE STORAGE MEDIUM

(71) Applicant: ZHEJIANG BRAIN-ENHANCE TECHNOLOGY CO. LTD, Hangzhou (CN)

(72) Inventors: Bicheng HAN, Hangzhou (CN); Max NEWLON, Hangzhou (CN); Lawrence FRANCHINI, Hangzhou (CN); Xiyujin HE, Hangzhou (CN)

(73) Assignee: ZHEJIANG BRAIN-ENHANCE TECHNOLOGY CO. LTD, Hangzhou (CN)

(21) Appl. No.: 17/365,991

(22) Filed: Jul. 1, 2021

Related U.S. Application Data
(63) Continuation of application No. PCT/CN2019/129358, filed on Dec. 27, 2019.

(30) Foreign Application Priority Data
Jan. 2, 2019 (CN) 201910003079.6

(51) Int. Cl. A61B 5/16; G16H 20/70; G16H 40/63; A61B 5/372; A61B 5/00

(52) U.S. Cl. CPC A61B 5/165; G16H 20/70; A61B 2505/09; A61B 5/372; A61B 5/6803; G16H 40/63

(57) ABSTRACT

Disclosed is a method for attention training, which includes: acquiring a first electroencephalogram (EEG) data that is currently detected through an EEG headband worn by a user; determining an attention level of the user, based on the first EEG data; and in response that the attention level is lower than a preset attention level, controlling to execute a first program based on the attention level, wherein the first program corresponds to a training plan. A device for attention training and a computer readable storage medium are further disclosed.

### CROSS REFERENCE TO RELATED APPLICATIONS

The present disclosure is a Continuation Application of International Application PCT/CN2019/129358, filed on Dec. 27, 2019, which claims priority to Chinese Patent Application titled “Method and device for attention training, and computer readable storage medium” filed in the National Intellectual Property Administration on Jan. 2, 2019 with application number 201910003079.6. The disclosures of the aforementioned applications are hereby incorporated by reference in their entireties.

### FIELD OF THE DISCLOSURE

The present disclosure relates to the technical field of electroencephalogram, in particular to a method and a device for attention training, and a computer readable storage medium.

### BACKGROUND OF THE DISCLOSURE

Sports build our body health, and are common ways to improve personal physique. A well-planned physical training may yield twice the efficiency of sports training.

In a process of events training, such as sports training, performance of the user would be negatively affected by the user failing to focus, and it may reduce the effect of training.

The aforementioned is assistant in understanding the technical solution of the present disclosure, and does not necessarily admit that the aforementioned constitutes the prior art.

### SUMMARY OF THE DISCLOSURE

The present disclosure is to provide a method and a device for attention training, and a computer readable storage medium, endeavoring to alleviate inattention of users and to improve effects of events training, for example of sports training accordingly.

Considering above, the present disclosure provides a method for attention training, which includes the following operations:

in response to receive a first detection instruction to detect a first electroencephalogram (EEG) data before an event, acquiring a first EEG data that is currently detected through an EEG headband worn by a user, where the first EEG data, and the EEG headband both correspond to the first detection instruction;

determining an attention level about current attention of the user, based on the first EEG data; and

in response that the attention level is lower than a preset attention level, controlling a first program based on the attention level, where the first program corresponds to a pre-event training plan.

Optionally, the operation of “controlling a first program based on the attention level” includes:

determining whether the first program is in operation;

if yes, acquiring an adjustment parameter for the first program based on the attention level;

adjusting a running parameter of the first program, based on the first adjustment parameter, and adjusting an output information of the first program.

Optionally, after the operation of “adjusting a running parameter of the first program, based on the first adjustment parameter”, the method further includes:

accumulating a first running time of the first program, after the running parameter is adjusted; and

in response that the first running time reaches a first preset time, continue to execute the operation of “acquiring a first EEG data that is currently detected through an EEG headband worn by a user”.

Optionally, after the operation of “determining whether the first program is in operation”, the method further includes:

if not, starting to run the first program based on the attention level;

in response that the first running time reaches a first preset time after starting the first program, continue to execute the operation of “acquiring a first EEG data that is currently detected through an EEG headband worn by a user”.

Optionally, after the operation of “determining an attention level about current attention of the user, based on the first EEG data”, the method further includes:

in response that the attention level is higher than or equal to a preset attention level, outputting prompt information to start a training.

Optionally, the method further includes:

in response to receive a second detection instruction to detect a second EEG data after an event, acquiring a second EEG data that is currently detected through an EEG headband worn by a user, where the user, and the EEG headband both correspond to the second detection instruction;

determining a relaxation level about current relaxation of the user, based on the second EEG data; and

in response to the relaxation level is lower than a preset relaxation level, controlling a second program based on the relaxation level, where the second program corresponds to a post-event recovery plan.

Optionally, the operation of “controlling a second program based on the relaxation level” includes:

acquiring a second adjustment parameter for the second program, based on the relaxation level; and

adjusting a running parameter of the second program, based on the second adjustment parameter, and adjusting output information of the second program.

Optionally, after the operation of “adjusting a running parameter of the second program, based on the second adjustment parameter”, the method further includes:

accumulating a second running time of the second program, after the running parameter is adjusted; and

in response that the second running time reaches a second preset time, continue to execute the operation of “acquiring a second EEG data that is currently detected through an EEG headband worn by a user”.

Additionally, the present disclosure further provides a device for attention training, where the device includes a memory, a processor and computer readable instructions stored in the memory and executable by the processor, and when executed by the processor, the computer readable instructions implement operations as described above.

Additionally, the present disclosure further provides a computer readable storage medium, where computer readable instructions are stored in the computer readable storage medium, and when the computer readable instructions are executed by a processor, operations are implemented as described above.

According to the present disclosure, a first EEG data that is currently detected is acquired through an EEG headband worn by a user, in response to receive a first detection instruction to detect a first EEG data before an event, where the first EEG data, and the EEG headband both correspond to the first detection instruction. an attention level is then determined about current attention of the user, based on the first EEG data. A first program is controlled based on the attention level in response that the attention level is lower than a preset attention level, where the first program corresponds to a pre-event training plan. User's attention is improved by controlling the first program based on the first EEG data, on condition that the user is not quite focused. The user would as such train himself with better focused attention, and the training performance and training effect can be improved.

### BRIEF DESCRIPTION OF THE DRAWINGS

FIG. 1 is a system schematic diagram showing a device for attention training in hardware operating environment according to some examples of the present disclosure.

FIG. 2 is a first flow chart of a method for attention training according to a first example of the present disclosure.

FIG. 3 is a second flow chart of a method for attention training according to a second and a third examples of the present disclosure.

FIG. 4 is a third flow chart of a method for attention training according to a fourth example of the present disclosure.

FIG. 5 is a fourth flow chart of a method for attention training according to a fifth example of the present disclosure.

FIG. 6 is a fifth flow chart of a method for attention training according to a sixth and a seventh examples of the present disclosure.

FIG. 7 is a sixth flow chart of a method for attention training according to another example of the present disclosure.

The implementation, functional characteristics and advantages of the present disclosure will be further described with reference to the attached drawings in combination with examples.

### DETAILED DESCRIPTION OF THE EMBODIMENTS

It should be understood that the specific examples described herein are merely for illustrative purpose and are not intended to limit the present disclosure.

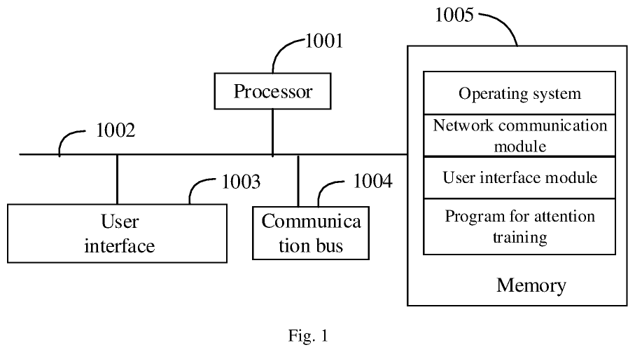

FIG. 1 shows a system schematic diagram regarding the device for attention training in hardware operating environment according to some examples of the present disclosure.

The device for attention training in the present disclosure can be a PC, a smart phone, a tablet computer, an electronic book reader, an MP3 (Moving Picture Experts Group Audio Layer III) player, an MP4 (moving picture experts group audio layer IV), video experts compress standard audio layer 3) player, a portable computer or another portable terminal device with display functions.

As shown in FIG. 1, the device for attention training may include a processor 1001, such as a CPU, a network interface 1004, a user interface 1003, a memory 1005, and a communication bus 1002. The communication bus 1002 is configured to realize the connection and communication between these components. The user interface 1003 may include a display and an input unit such as a keyboard, and the optional user interface 1003 may also include a standard wired interface and a wireless interface. The network interface 1004 may optionally include a standard wired interface and a wireless interface (such as a WI-FI interface). The memory 1005 may be a high-speed RAM memory or a non-volatile memory, such as a disk memory. Optionally, the memory 1005 may also be a storage device independent of the aforementioned processor 1001.

Optionally, the device for attention training may also include a camera, a radio frequency (RF) circuit, a sensor, an audio circuit, a WiFi module, and the like. where, sensors can be light sensors, motion sensors and the like.

It can be understood by those skilled in the art that the structure of the device for attention training shown in FIG. 1 does not constitute a limitation on the device for attention training, and may include more or fewer components than shown, or combine some components, or arrange different components.

As shown in FIG. 1, the memory 1005 as a computer storage medium may include an operating system, a network communication module, a user interface module, and computer programs.

In the components of the device shown in FIG. 1, the network interface 1004 is mainly configured to connect with a back-end server and perform data communication with the back-end server. The user interface 1003 is mainly configured to connect the client (user end) and perform data communication with the client end; while the processor 1001 can be configured to call the computer readable programs stored in the memory 1005.

In some implementations, the device for attention training includes a memory 1005, a processor 1001, and computer readable instructions stored in the memory 1005 and executable by the processor 1001. When the processor 1001 calls the computer readable instructions stored in the memory 1005, it implements the following operations:

in response to receive a first detection instruction to detect a first EEG data before an event, acquiring a first EEG data that is currently detected through an EEG headband worn by a user, where the first EEG data, and the EEG headband both correspond to the first detection instruction;

determining an attention level about current attention of the user, based on the first EEG data; and

in response that the attention level is lower than a preset attention level, controlling a first program based on the attention level, where the first program corresponds to a pre-event training plan.

Further, the processor 1001 may call the programs stored in the memory 1005, and implement the following operations:

determining whether the first program is in operation;

if yes, acquiring an adjustment parameter for the first program based on the attention level;

adjusting a running parameter of the first program, based on the first adjustment parameter, and adjusting output information of the first program.

Further, the processor 1001 may call the programs stored in the memory 1005, and implement the following operations:

accumulating a first running time of the first program, after the running parameter is adjusted; and

in response that the first running time reaches a first preset time, continue to execute the operation of “acquiring a first EEG data that is currently detected through an EEG headband worn by a user”.

Further, the processor 1001 may call the programs stored in the memory 1005, and implement the following operations:

if not, starting to run the first program based on the attention level; and

in response that the first running time reaches a first preset time after starting the first program, continue to execute the operation of “acquiring a first EEG data that is currently detected through an EEG headband worn by a user”.

Further, the processor 1001 may call the programs stored in the memory 1005, and implement the following operations:

in response that the attention level is higher than or equal to a preset attention level, outputting prompt information to start a training.

Further, the processor 1001 may call the programs stored in the memory 1005, and implement the following operations:

in response to receive a second detection instruction to detect a second EEG data after an event, acquiring a second EEG data that is currently detected through an EEG headband worn by a user, where the second EEG data, and the EEG headband both correspond to the second detection instruction;

determining a relaxation level about current relaxation of the user, based on the second EEG data; and

in response to the relaxation level is lower than a preset relaxation level, controlling a second program based on the relaxation level, where the second program corresponds to a post-event recovery plan.

Further, the processor 1001 may call the programs stored in the memory 1005, and implement the following operations:

acquiring a second adjustment parameter for the second program, based on the relaxation level; and

adjusting a running parameter of the second program, based on the second adjustment parameter, and adjusting output information of the second program.

Further, the processor 1001 may call the programs stored in the memory 1005, and implement the following operations:

accumulating a second running time of the second program, after the running parameter is adjusted; and

in response that the second running time reaches a second preset time, continue to execute the operation of “acquiring a second EEG data that is currently detected through an EEG headband worn by a user”.

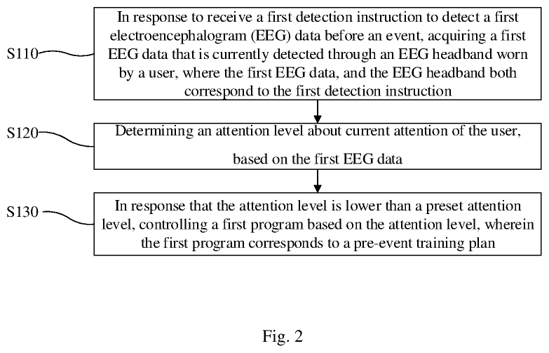

The disclosure further provides a method for attention training. Referring to FIG. 2, a flow chart is shown regarding the first example of the method for attention training.

Specifically, the method for attention training includes:

Operation S110, in response to receive a first detection instruction to detect a first EEG data before an event, acquiring a first EEG data that is currently detected through an EEG headband worn by a user, where the first EEG data, and the EEG headband both correspond to the first detection instruction.

Where the device can be PC or another equipment, which can establish communication with at least one EEG headband, and the owner of the EEG headband detects the EEG signal of the user (wearer), and sends the currently detected EEG signal to the device for attention training.

The user can train before the event such as a sports event and improve the user's attention according to the pre-event training plan. The EEG signal of the user who wears the EEG headband is detected through the EEG headband to determine the user's current attention. Specifically, the EEG headband can be provided with a button for detecting user's attention before an event even starts, which can trigger the first detection instruction to detect the pre-event EEG data. Or the user can trigger the first detection instruction to detect the pre-event EEG data via a display interface of the device.

In the present example, when receiving the first detection instruction for detecting the pre-event EEG data, the first EEG data corresponding to the first detection instruction is acquired through the EEG headband which also corresponds to the first detection instruction. Specifically, an acquisition request is sent to the EEG headband corresponding to the first detection instruction, and the EEG headband sends the first EEG data that is detected at current time point to the device, where the first EEG data can include the EEG data of the user who wears the EEG headband within a preset time interval from the current time point.

It should be noted that one or more EEG headbands can be bound with the device. When receiving the first detection instruction for detecting the EEG data before an event, the device firstly determines which EEG headband that corresponds to the first detection instruction, and further judges whether the EEG headband is in a working state, that is, whether the user is wearing the EEG headband at present. If yes, the headband acquires first EEG data currently detected of the user. Otherwise, an alarm message would be prompted that the first EEG data cannot be acquired, so as to indicate the user to wear the EEG headband or indicate the user to check whether the EEG headband is started.

Operation S120, determining an attention level about current attention of the user, based on the first EEG data.

Specifically, after acquiring the first EEG data, the attention level corresponding to the user's current attention is determined based on the first EEG data. Specifically, this attention level corresponding to the first EEG data can be calculated according to the existing algorithm, that is, the attention level corresponding to the user's current attention is calculated, and the calculated attention level which corresponds to the current attention of the user, also corresponds to the first detection instruction.

Operation S130, in response that the attention level is lower than a preset attention level, controlling a first program based on the attention level, where the first program corresponds to a pre-event training plan.

In the present example, when the attention level is acquired, it is judged or determined whether the attention level is less than a preset attention level, and if the attention level is less than the preset attention level, the first program corresponding to the user's pre-event training plan is controlled based on the attention level. Information is further outputted for the first program, which is used to enhance the user's attention. The information outputted includes audio information and/or video information. The device may adjust the first program according to the attention level, and further adjusts the output information. For example, the volume of the outputted audio information can be increased, and the brightness, contrast and saturation of the outputted video information can be enhanced, in order to enhance the user's attention by watching the adjusted video or by listening to the adjusted audio.

The preset attention level is a typical attention level when the user is focused, and when the attention level of the user is higher than or equal to the preset attention level, it indicates that the user is currently in a state of concentration or in a focused state.

A plurality of focus ranges can be preset, the maximum values of the preset focus ranges are all lower than the preset attention level, and each preset focus range corresponds to one group of adjustment parameters for the first program.

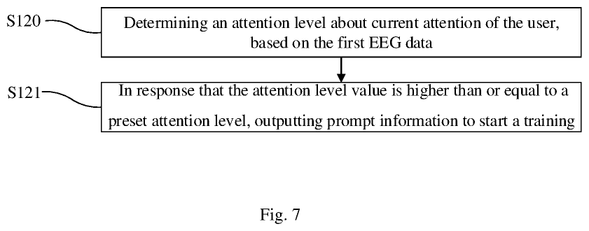

Furthermore, in some example, FIG. 7 is referred to, after operation S120, the method for attention training further includes:

Operation S121, in response that the attention level is higher than or equal to a preset attention level, outputting prompt information to start a training.

When the attention level is higher than or equal to the preset attention level, the user is currently in a state of concentration. So prompt information can be outputted, indicating that the user can start the training, for example a sports training. The user will then perform sports training with his attention concentrated, thereby improving training performance and training effect of the user.

According to the method described above, a current EEG data of a user is acquired through an EEG headband, in response to receive a first detection instruction to detect a first EEG data before an event, where the first EEG data, and the EEG headband both correspond to the first detection instruction. an attention level is then determined about current attention of the user, based on the first EEG data. A first program is controlled based on the attention level in response that the attention level is lower than a preset attention level, where the first program corresponds to a pre-event training plan. User's attention is improved by controlling the first program based on the first EEG data, when the user is not well focused. The user would as such train himself under focused attention, and the training performance and training effect can be improved.

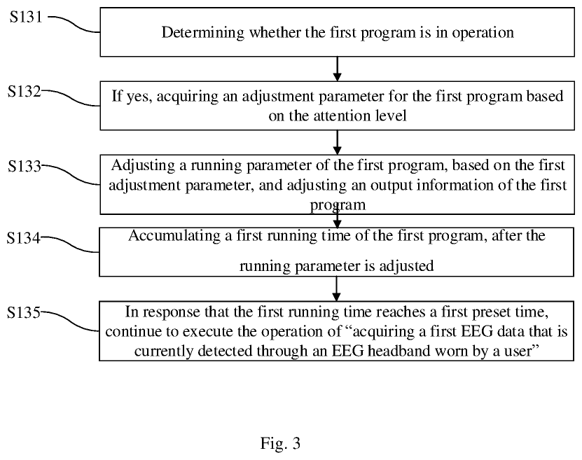

Based on the first example, a second example of the method for attention training of the present disclosure is proposed. In the present example, FIG. 3 is referred to, operation S130 includes:

operation S131, determining whether the first program is in operation;

operation S132, if yes, acquiring an adjustment parameter for the first program based on the attention level; and

operation S133, adjusting a running parameter of the first program, based on the first adjustment parameter, and adjusting an output information of the first program.

In the present example, if the attention level is less than the preset attention level, it will be then judged or determined whether the first program is in a running state or is operating at present. And if the first program is in a running state, the adjustment parameters for the first program will be acquired based on the attention level. Specifically, a plurality of preset focus ranges can be preset, the maximum values of the preset focus ranges are all lower than the preset attention level, and each preset focus range corresponds to one group of adjustment parameters for the first program. When the first program is running, it will be determined which preset focus range the current attention level belongs to, and the group of adjustment parameters which corresponds to the determined preset focus range is acquired, so that the running parameters of the first program will be adjusted based on these adjustment parameters. Further, the output information of the first program is therefore adjusted. For example, an increase in the volume of the output audio information, or an improvement of the brightness, contrast and saturation of the output video information is performed. As such, the user will watch the adjusted video or listen to the adjusted audio.

According to the method for attention training proposed in the present example, it first judges whether the first program is in a running state. Then, if yes, it acquires adjustment parameters for the first program based on the attention level. Then it adjusts the running parameters of the first program based on the adjustment parameters, so as to adjust the output information of the first program. An accurate adjustment of the running parameters of the first program is performed as a result. The method accurately controls the first program according to the first EEG data, improves the attention of users by the output information of the first program, and further enables the user to carry out the training in a focused state, so as to improve the sports performance and training effect of training for the user.

Based on the second embodiment, a third example of the method for attention training of the present disclosure is proposed. In the present example, FIG. 3 is referred to, after operation S133, the method further includes:

operation S134, accumulating a first running time of the first program, after the running parameter is adjusted; and

operation S135, in response that the first running time reaches a first preset time, continue to execute the operation of “acquiring a first EEG data that is currently detected through an EEG headband worn by a user”.

In the present example, after adjusting the running parameters of the first program, the first running time of the first program is accumulated. And when the first running time reaches the first preset time, operation S110 is recirculated to realize the cyclic control to the first program for detecting the attention of the user.

According to the method proposed by the present example, the first running time of the first program is accumulated after adjusting the running parameters. Then execution of the operation of “acquiring a first EEG data that is currently detected through an EEG headband worn by a user” is restarted, when the first running time reaches the first preset time. As such, the first program can be adjusted at a regular frequency according to the EEG data of the user, and the first program can be adjusted by the regular fed-back EEG data to improve the user's attention.

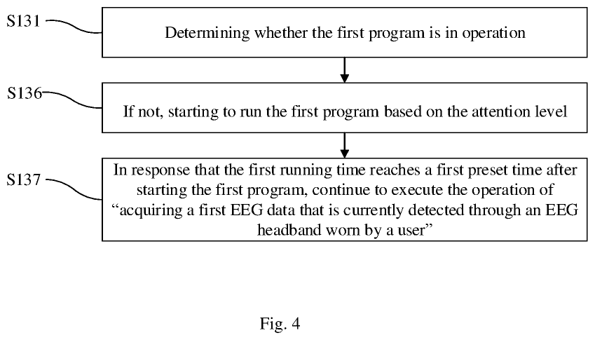

Based on the second example, a fourth example of the method for attention training of the present disclosure is proposed. In the present example, FIG. 4 is referred to, after operation S131, the method further includes:

operation S136, if not, starting to run the first program based on the attention level; and

operation S137, in response that the first running time reaches a first preset time after starting the first program, continue to execute the operation of “acquiring a first EEG data that is currently detected through an EEG headband worn by a user”.

In the present example, if the attention level is less than the preset attention level, it is judged whether the first program is running. And if it is not currently running, the first program is started and run based on the attention level. Specifically, the first program can be started first, its adjustment parameters are then acquired based on the attention level. The output information of the first program will then be adjusted, for example, by increasing the volume of the output audio information and/or improving the brightness, contrast, saturation, etc. of the output video information. The attention of users is as a consequence, enhanced by watching the adjusted video information or listening to the adjusted audio information.

Specifically, a plurality of preset focus ranges can be preset, the maximum values of these preset focus ranges are all lower than the preset attention level, and each preset focus range corresponds to one group of adjustment parameters for the first program. When the first program is in a running state, the preset focus range to which the attention level belongs is determined or judged, and the adjustment parameters corresponding to the preset focus range to which the attention level belongs are acquired.

According to the method for attention training proposed in the present example, if the first program is not currently running, the first program will be started and run based on the attention level. When the running time of the first program reaches the first preset time, execution of the operation of “acquiring a first EEG data that is currently detected through an EEG headband worn by a user” would be circulated. The first program is initiated based on the attention level, and is adjusted based on the EEG data of the user, where the EEG data is fed back at regular intervals. Therefore, the attention of the user is improved.

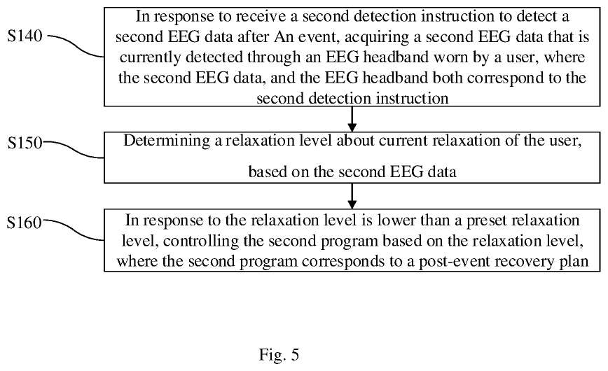

Based on the above examples, a fifth example of the method for attention training of the present disclosure is proposed. In the present example, FIG. 5 is referred to, the method further includes:

operation S140, in response to receive a second detection instruction to detect a second EEG data after an event, acquiring a second EEG data that is currently detected through an EEG headband worn by a user, where the second EEG data, and the EEG headband both correspond to the second detection instruction;

operation S150, determining a relaxation level about current relaxation of the user, based on the second EEG data; and

operation S160, in response to the relaxation level is lower than a preset relaxation level, controlling the second program based on the relaxation level, where the second program corresponds to a post-event recovery plan.

The user can perform recovery training after an event according to the post-event training plan, to smooth and calm down the user's spirits. The EEG signal of the user who wears the EEH headband is detected through the EEG headband to determine the user's current relaxation level. Specifically, the EEG headband can be provided with a button which can trigger the second detection instruction to detect the post-event EEG data that reflects the user's relaxation. Or the user can trigger the second detection instruction to detect the post-event EEG data via a display interface of the device.

In the present example, when receiving the second detection instruction for detecting the post-event EEG data, the second EEG data corresponding to the second detection instruction is acquired through the EEG headband which also corresponds to the second detection instruction. Specifically, an acquisition request is sent to the EEG headband corresponding to the second detection instruction, and the EEG headband sends the second EEG data that is detected at current time point to the device, where the second EEG data can include the EEG data of the user who wears the EEG headband within a preset time interval from the current time point.

In the present example, after acquiring the second EEG data, the relaxation level corresponding to the user's current relaxation is determined based on the second EEG data. Specifically, the relaxation level corresponding to the second EEG data can be calculated according to the existing algorithm, that is, the relaxation level corresponding to the user's current relaxation is calculated, and the calculated relaxation level which corresponds to the current relaxation of the user, also corresponds to the second detection instruction.

In the present example, when the relaxation level is acquired, it is judged whether the relaxation level is less than the preset relaxation level, and if the relaxation level is less than the preset relaxation level, the second program corresponding to the user's post-event recovery plan is controlled according to the relaxation level. And the output information of the second program is used to help the user to relax, which includes audio information and/or video information and can be adjusted according to the relaxation level. The output information of the second program is adjusted as such, for example, by increasing the volume of the output audio information, or by improving the brightness, contrast and saturation of the output video information, so as to accelerate the user's relaxation by watching the adjusted video information or listening to the adjusted audio information.

The preset relaxation level is a threshold value when the user's relaxation reaches a certain degree. When the user's relaxation level is higher than or equal to the preset relaxation level, the user is currently in a relatively relaxed state.

Furthermore, in some example, after operation S150, the method for attention training further includes:

when the relaxation level is higher than or equal to the preset relaxation level, outputting prompt information that the user is in a relaxed state, and controlling the second program according to the current running parameters. When accumulated running time reaches the second preset time, the operation of “acquiring a second EEG data that is currently detected through an EEG headband worn by a user” is circulated.

It should be noted that if the relaxation level is less than the preset relaxation level and the second program is not started, then the second program corresponding to the user's post-event recovery plan is controlled based on the relaxation level.

According to the method described above, a current EEG data of a user is acquired through an EEG headband, in response to receive a second detection instruction to detect a second EEG data after an event, where the second EEG data, and the EEG headband both correspond to the second detection instruction. A relaxation level is then determined about current relaxation of the user, based on the second EEG data. A second program is controlled based on the relaxation level in response that the relaxation level is lower than a preset relaxation level, where the second program corresponds to a post-event training plan. User's feelings are smoothed by controlling the second program based on the second EEG data, when the user is not well relaxed. The method helps the user to relax himself in mind, and remain harmonized in body and soul.

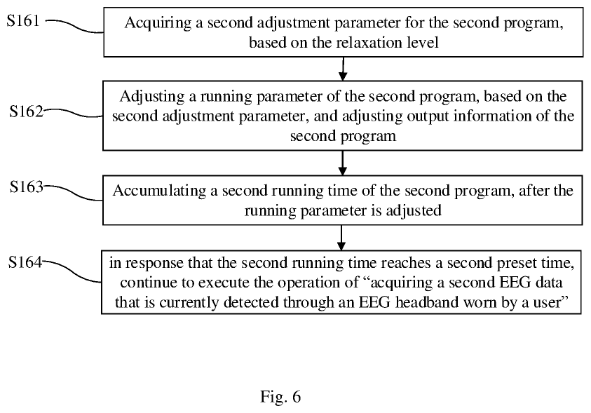

Based on the fifth embodiment, a sixth example of the method for attention training of the present disclosure is proposed. In the present example, FIG. 6 is referred to, operation S160 includes:

operation S161, acquiring a second adjustment parameter for the second program, based on the relaxation level.

operation S162, adjusting a running parameter of the second program, based on the second adjustment parameter, and adjusting output information of the second program.

A plurality of relaxation ranges can be preset, the maximum values of the preset relaxation ranges are all lower than the preset relaxation level, and each preset relaxation range corresponds to one group of adjustment parameters for the second program.

In the present example, if the relaxation level is less than the preset relaxation level, and if the second program is in a running state, the adjustment parameters for the second program will be acquired based on the relaxation level. Specifically, a plurality of preset relaxation ranges can be preset, the maximum value of each relaxation ranges is less than the preset relaxation level, and each preset relaxation range corresponds to one group of adjustment parameters for the second program. When the second program is running, it will be determined which preset relaxation range the current relaxation level belongs to, and the group of adjustment parameters which corresponds to the determined preset relaxation range is acquired, so that the running parameters of the second program will be adjusted based on these adjustment parameters. Further, the output information of the second program is therefore adjusted. For example, an increase in the volume of the output audio information, or an improvement in the brightness, contrast and saturation of the output video information is performed. As such, the user will watch the adjusted video or listen to the adjusted video.

The method for attention training proposed in this example obtains the second adjustment parameters for the second program based on the relaxation level.

The running parameters of the second program are adjusted based on the second adjustment parameters, so as to adjust the output information of the second program, accurately control the second program according to the second EEG data. It helps to improve the user's relaxation degree by the output information of the second program, and then guide the user to relax and calm down through the sound or picture interacted with the user, so that the user makes his nerves relaxed and his mind relieved.

Based on the sixth example, a seventh example of the method for attention training of the present disclosure is proposed. In the present example, FIG. 6 is referred to, after operation S162, the method further includes:

operation S163, accumulating a second running time of the second program, after the running parameter is adjusted; and

operation S164, in response that the second running time reaches a second preset time, continue to execute the operation of “acquiring a second EEG data that is currently detected through an EEG headband worn by a user”.

In the present example, after adjusting the running parameters of the second program, the second running time of the second program is accumulated, and when the second running time reaches the second preset time, operation S140 is recirculated to realize the cyclic control of the second program for detecting the attention of the user. The user may relax and calm down through the sound or picture interacted with the user, so that the user makes his nerves relaxed and his mind relieved.

According to the method proposed by the present example, the second running time of the second program is accumulated after adjusting the running parameters. Then execution of the operation of “acquiring a second EEG data that is currently detected through an EEG headband worn by a user” is restarted, when the second running time reaches the second preset time. The second program can be adjusted at a regular frequency according to the EEG data of the user, and the second program can be adjusted by the regular fed-back EEG data to improve the user's relaxation.

Furthermore, the present disclosure proposes a computer readable storage medium, where computer readable instructions are stored in the computer readable storage medium, and when the computer readable instructions are executed by a processor, following operations are implemented:

in response to receive a first detection instruction to detect a first EEG data before an event, acquiring a first EEG data that is currently detected through an EEG headband worn by a user, where the first EEG data, and the EEG headband both correspond to the first detection instruction;

determining an attention level about current attention of the user, based on the first EEG data; and

in response that the attention level is lower than a preset attention level, controlling a first program based on the attention level, where the first program corresponds to a pre-event training plan.

Further, when the computer readable instructions are executed by the processor, the following operations are further implemented:

determining whether the first program is in operation;

if yes, acquiring an adjustment parameter for the first program based on the attention level; and

adjusting a running parameter of the first program, based on the first adjustment parameter, and adjusting output information of the first program.

Further, when the computer readable instructions are executed by the processor, the following operations are further implemented:

accumulating a first running time of the first program, after the running parameter is adjusted; and

in response that the first running time reaches a first preset time, continue to execute the operation of “acquiring a first EEG data that is currently detected through an EEG headband worn by a user”.

Further, when the computer readable instructions are executed by the processor, the following operations are further implemented:

if not, starting to run the first program based on the attention level; and

in response that the first running time reaches a first preset time after starting the first program, continue to execute the operation of “acquiring a first EEG data that is currently detected through an EEG headband worn by a user”.

Further, when the computer readable instructions are executed by the processor, the following operations are further implemented:

in response that the attention level is higher than or equal to a preset attention level, outputting prompt information to start a training.

Further, when the computer readable instructions are executed by the processor, the following operations are further implemented:

in response to receive a second detection instruction to detect a second EEG data after an event, acquiring a second EEG data that is currently detected through an EEG headband worn by a user, where the user, and the EEG headband both correspond to the second detection instruction;

determining a relaxation level about current relaxation of the user, based on the second EEG data; and

in response to the relaxation level is lower than a preset relaxation level, controlling the second program based on the relaxation level, where the second program corresponds to a post-event recovery plan.

Further, when the computer readable instructions are executed by the processor, the following operations are further implemented:

acquiring a second adjustment parameter for the second program, based on the relaxation level; and

adjusting a running parameter of the second program, based on the second adjustment parameter, and adjusting an output information of the second program.

Further, when the computer readable instructions are executed by the processor, the following operations are further implemented:

accumulating a second running time of the second program, after the running parameter is adjusted; and

in response that the second running time reaches a second preset time, continue to execute the operation of “acquiring a second EEG data that is currently detected through an EEG headband worn by a user”.

It should be noted that in this document, the terms “comprising” “including” or any other variation thereof are intended to cover a non-exclusive inclusion, such that a process, method, article, or system that includes a list of elements includes not only those elements but also other elements not expressly listed, or elements inherent to such process, method, article, or system. Without further restrictions, an element defined by the statement “includes an” does not exclude the presence of another identical element in a process, method, article, or system including the element.

The aforementioned serial numbers regarding the examples of the present disclosure are for description only and do not represent the superiority and inferiority of the examples.

From the above description of the examples, those skilled in the art can clearly understand that the method of the above examples can be implemented by means of software plus necessary general-purpose hardware platforms. Of course, it can also be implemented by means of hardware, but in many cases the former is a better embodiment. Based on this understanding, the technical solution of the present disclosure, in essence, or the part contributing to the prior art, can be embodied in the form of a software product stored in a storage medium (such as ROM/RAM, magnetic disk, diskette) as described above, including several instructions to cause a terminal device (which can be a mobile phone, computer, server, air conditioner, or network device, etc.) to perform the methods described in various examples of the present disclosure.

The description aforementioned is only the optional example of the present disclosure and is not intended to limit the scope of the present disclosure. Any equivalent structural or flow modification made by using the description and drawings of the present disclosure or direct/indirect application in other related technical fields under the concept of the present disclosure shall be included in the claimed scope of the present disclosure.

What is claimed is:

1. A method for attention training, comprising:
acquiring a first electroencephalogram (EEG) data that is currently detected through an EEG headband worn by a user;
determining an attention level of the user, based on the first EEG data; and
in response that the attention level is lower than a preset attention level, controlling to execute a first program based on the attention level, wherein the first program corresponds to a training plan.

2. The method according to claim 1, wherein the operation of “controlling to execute a first program based on the attention level” comprises:
determining that the first program is in operation, acquiring a first adjustment parameter for the first program based on the attention level; and
adjusting a running parameter of the first program, based on the first adjustment parameter, and adjusting output information of the first program.

3. The method according to claim 2, wherein after the operation of “adjusting a running parameter of the first program based on the first adjustment parameter”, the method further comprises:
accumulating a first running time of the first program, after the running parameter is adjusted; and
in response that the first running time reaches a first preset time, continuing to execute the operation of “acquiring a first EEG data that is currently detected through an EEG headband worn by a user”.

4. The method according to claim 2, wherein the operation of “controlling to execute a first program based on the attention level” comprises:
determining that the first program is not in operation, starting to run the first program based on the attention level; and
in response that a running time reaches a first preset time after starting the first program, continue to execute the operation of “acquiring a first EEG data that is currently detected through an EEG headband worn by a user”.

5. The method according to claim 1, wherein after the operation of “determining an attention level about current attention of the user, based on the first EEG data”, the method further comprises:
in response that the attention level is higher than or equal to a preset attention level, outputting prompt information to start a training.

6. The method according to claim 1, further comprising:
in response to receive a second detection instruction to detect a second EEG data, acquiring a second EEG data that is currently detected through a second EEG headband worn by the user, wherein the second EEG data, and the second EEG headband both correspond to the second detection instruction;
determining a relaxation level of the user, based on the second EEG data; and
in response that the relaxation level is lower than a preset relaxation level, controlling to execute the second program based on the relaxation level, wherein the second program corresponds to a recovery plan.

7. The method according to claim 6, wherein the operation of “controlling to execute the second program based on the relaxation level” comprises:
acquiring a second adjustment parameter for the second program, based on the relaxation level; and
adjusting a running parameter of the second program, based on the second adjustment parameter, and adjusting output information of the second program.

8. The method according to claim 7, wherein after the operation of “adjusting a running parameter of the second program, based on the second adjustment parameter”, the method further comprises:
accumulating a second running time of the second program, after the running parameter is adjusted; and
in response that the second running time reaches a second preset time, continue to execute the operation of “acquiring a second EEG data that is currently detected through a second EEG headband worn by the user”.

9. The method according to claim 2, further comprising:
in response to receive a second detection instruction to detect a second EEG data, acquiring a second EEG data that is currently detected through a second EEG headband worn by the user, wherein the second EEG data, and the second EEG headband both correspond to the second detection instruction;
determining a relaxation level about current relaxation of the user, based on the second EEG data; and
in response to the relaxation level is lower than a preset relaxation level, controlling to execute the second program based on the relaxation level, wherein the second program corresponds to a recovery plan.

10. The method according to claim 9, wherein the operation of “controlling to execute the second program based on the relaxation level” comprises:
acquiring a second adjustment parameter for the second program, based on the relaxation level; and
adjusting a running parameter of the second program, based on the second adjustment parameter, and adjusting output information of the second program.

11. The method according to claim 10, wherein after the operation of “adjusting a running parameter of the second program, based on the second adjustment parameter”, the method further comprises:
accumulating a second running time of the second program, after the running parameter is adjusted; and
in response that the second running time reaches a second preset time, continue to execute the operation of “acquiring a second EEG data that is currently detected through a second EEG headband worn by the user”.

12. The method according to claim 3, further comprising:
in response to receive a second detection instruction to detect a second EEG data, acquiring a second EEG data that is currently detected through a second EEG headband worn by the user, wherein the second EEG data, and the second EEG headband both correspond to the second detection instruction;
determining a relaxation level of the user, based on the second EEG data; and
in response to the relaxation level is lower than a preset relaxation level, controlling to execute the second program based on the relaxation level, wherein the second program corresponds to a recovery plan.

13. The method according to claim 12, wherein the operation of “controlling to execute to execute the second program based on the relaxation level” comprises:
acquiring a second adjustment parameter for the second program, based on the relaxation level; and
adjusting a running parameter of the second program, based on the second adjustment parameter, and adjusting output information of the second program.

14. The method according to claim 13, wherein after the operation of “adjusting a running parameter of the second program, based on the second adjustment parameter”, the method further comprises:
accumulating a second running time of the second program, after the running parameter is adjusted; and
in response that the second running time reaches a second preset time, continue to execute the operation of “acquiring a second EEG data that is currently detected through a second EEG headband worn by the user”.

15. The method according to claim 4, further comprising:
in response to receive a second detection instruction to detect a second EEG data, acquiring a second EEG data that is currently detected through a second EEG headband worn by the user, wherein the second EEG data, and the second EEG headband both correspond to the second detection instruction;
determining a relaxation level about current relaxation of the user, based on the second EEG data; and
in response to the relaxation level is lower than a preset relaxation level, controlling to execute the second program based on the relaxation level, wherein the second program corresponds to a recovery plan.

16. The method according to claim 15, wherein the operation of “controlling to execute the second program based on the relaxation level” comprises:
acquiring a second adjustment parameter for the second program, based on the relaxation level; and
adjusting a running parameter of the second program, based on the second adjustment parameter, and adjusting output information of the second program.

17. The method according to claim 16, wherein after the operation of “adjusting a running parameter of the second program, based on the second adjustment parameter”, the method further comprises:
accumulating a second running time of the second program, after the running parameter is adjusted; and
in response that the second running time reaches a second preset time, continue to execute the operation of “acquiring a second EEG data that is currently detected through a second EEG headband worn by the user”.

18. The method according to claim 5, further comprising:
in response to receive a second detection instruction to detect a second EEG data, acquiring a second EEG data that is currently detected through a second EEG headband worn by the user, wherein the second EEG data, and the second EEG headband both correspond to the second detection instruction;
determining a relaxation level of the user, based on the second EEG data; and
in response to the relaxation level is lower than a preset relaxation level, controlling to execute the second program based on the relaxation level, wherein the second program corresponds to a recovery plan.

19. A device for attention training, wherein the device comprises a memory, a processor and computer readable instructions stored on the memory and executable on the processor, and when executed by the processor, the computer readable instructions implement following operations:
acquiring a first EEG data that is currently detected through an EEG headband worn by a user;
determining an attention level about current attention of the user, based on the first EEG data; and
in response that the attention level is lower than a preset attention level, controlling to execute a first program based on the attention level, wherein the first program corresponds to a training plan.

20. A computer readable storage medium, wherein computer readable instructions are stored on the computer readable storage medium, and when the computer readable instructions are executed by a processor, following operations are implemented:
acquiring a first EEG data that is currently detected through an EEG headband worn by a user;
determining an attention level of the user, based on the first EEG data; and
in response that the attention level is lower than a preset attention level, controlling to execute a first program based on the attention level, wherein the first program corresponds to a training plan.
